(19) United States
(12) Patent Application Publication
(10) Pub. No.: US 2023/0121899 A1
(43) Pub. Date: Apr. 20, 2023

(54) AUTOMATIC REGISTRATION OF INTRAORAL SURFACE AND VOLUMETRIC SCAN DATA FOR ORTHODONTIC TREATMENT PLANNING

(71) Applicant: CVSTOM Co., San Francisco, CA (US)

(72) Inventors: Anton NAUMOVETS, Kiev (UA); Kjeld A. AAMODT, San Francisco, CA (US)

(21) Appl. No.: 17/938,525

(22) Filed: Oct. 6, 2022

Related U.S. Application Data
(60) Provisional application No. 63/256,891, filed on Oct. 18, 2021.

(51) Int. Cl. A61C 7/00; A61C 7/08; A61C 9/00

(52) U.S. Cl. CPC A61C 7/002; A61C 7/08; A61C 9/0053; A61C 2007/004

(57) ABSTRACT

A method for automatic registration of dental image data comprises receiving three-dimensional intraoral surface scan data of a dentition of a patient, receiving three-dimensional volumetric scan data of the dentition, generating a first set of descriptors characterizing tooth crown surfaces of the dentition from the intraoral surface scan data, generating a second set of descriptors characterizing tooth crown surfaces of the dentition from the volumetric scan data, determining a best-fit registration transform based on the first and second sets of descriptors, and aligning the intraoral surface scan data and the volumetric scan data based on the best-fit registration transform.

### CROSS-REFERENCE TO RELATED APPLICATIONS

This application claims priority to U.S. Patent Application Ser. No. 63/256,891 filed Oct. 18, 2021, which is hereby incorporated in its entirety by this reference.

### TECHNICAL FIELD

This invention relates generally to the field of orthodontic treatment planning.

### BACKGROUND

Orthodontia is a specialty of dentistry that aims to correct a patient's teeth and jaws that are improperly positioned, such as for health and/or cosmetic reasons. Generally, orthodontic treatments leverage the application of external forces (from orthodontic devices) to cause the progressive movement of one or more teeth from their original improper positions to desired positions.

The efficiency of orthodontic tooth movement is directly influenced, in part, by the accuracy and precision of the orthodontic treatment plan as it relates to the patient's unique, individual condition. In conventional orthodontia, accuracy and precision of the orthodontic treatment plan is dependent upon the experience and/or capability of the particular treating doctor. Furthermore, treatment success is largely dependent upon how well the locations of the tooth crown, tooth root, and jawbones are defined at the time of treatment initiation, as well as how well the tooth movement over time throughout treatment is modeled. However, variability in skill levels of the treating doctor, other clinicians, and orthodontic device manufacturers results in variable treatment outcomes. As such, this results in a historically slow and burdensome treatment process for patients, because orthodontic appliances require high amounts of guesswork and adjustments, and/or incorporate error and noise that must be overcome through multiple in-person adjustment and refine visits, all of which add costs and burden to treatment.

Effective orthodontic treatment planning can incorporate intraoral surface scan data and volumetric scan data, which contain different sets of data that are helpful to enable precise and efficient orthodontic treatment planning. For example, an intraoral surface scan may provide high precision data for teeth crown and gum surfaces, while a volumetric scan provides data related to teeth root orientation, cranial and jawbone volume and density. To model orthodontic tooth movement accurately, these two sets of data can be aligned and combined. Current techniques to perform such combination typically requires either a user to place markers at anatomical landmarks on both sets of data, or manually adjust the orientation of one or both scan images, or both. However, both of these techniques are time-consuming and require manual input, which is also subject to risk of error and subjectivity.

Accordingly, there is a need for new and improved methods and systems for defining a patient's relevant anatomy (e.g., tooth crown, tooth root, jawbones) before treatment is initiated, so that further orthodontic treatment planning may proceed in an accurate, precise, and efficient manner.

### SUMMARY

Generally, in some variations, a method for automatic registration of dental image data may include receiving three-dimensional intraoral surface scan data of a dentition of a patient, receiving three-dimensional volumetric scan data of the dentition, generating a first set of descriptors characterizing tooth crown surfaces of the dentition from the intraoral surface scan data, generating a second set of descriptors characterizing tooth crown surfaces of the dentition from the volumetric scan data, determining a best-fit registration transform based on the first and second sets of descriptors, and aligning the intraoral surface scan data and the volumetric scan data based on the best-fit registration transform. In some variations, the intraoral surface scan data may include optical color scan data and the volumetric scan data may include CBCT X-ray scan data.

In some variations, at least one of the first set of descriptors and the second set of descriptors may include one or more descriptors characterizing curvature of the tooth crown surfaces. For example, such one or more descriptors may include an estimate of a local surface normal direction, a principal curvature direction, a principal curvature value, or any combination thereof, at each of the plurality of points of the tooth crown surfaces. In some variations, a descriptor may include an estimate of a local surface normal direction, two principal curvature directions, two principal curvature values associated with the two principal curvature directions, at each of the plurality of points on the tooth crown surfaces.

In some variations, determining the best-fit registration transform may include generating one or more registration transform candidates based on matching descriptors from the first and second sets of descriptors. One or more of the registration transform candidates may be generated based on a voting scheme such as a Hough transform. Furthermore, determining the best-fit registration transform may include generating one or more refined registration candidates by applying an iterative local registration procedure to the one or more registration transform candidates. Various suitable techniques to generate the refined registration candidate(s) may be used, including, for example, an iterative closest point to plane algorithm or an iterative closest point to point algorithm. Additionally or alternatively, in some variations, determining the best-fit registration transform may include generating a surface proximity measure associated with each of the one or more refined registration candidates, and identifying the refined registration transform candidate associated with a lowest surface proximity measure. In some variations, the best-fit registration transform may be determined by generating a surface proximity measure associated with each of the registration transform candidates (e.g., not the refined registration candidates generated from an iterative local registration procedure).

In some variations, the intraoral surface scan data and the volumetric scan data may be aligned based on the best-fit registration transform. For example, the method may include aligning the intraoral surface scan data and the volumetric scan data by applying the best-fit registration transform to the intraoral surface scan data to generate a transformed intraoral surface scan image (e.g., by applying the best-fit registration transform to a triangulated surface of the intraoral surface scan data).

In some variations, the method may include determining and applying separate best-fit registration transforms for separate portions of scan data. For example, the method may include determining one best-fit registration transform for an upper jaw of the patient (where this best-fit registration transform is configured to transform intraoral surface scan data of dentition on the upper jaw of the patient) and determining another best-fit registration transform for a lower jaw of the patient (where this best-fit registration transform is configured to transform intraoral surface scan data of dentition on the lower jaw of the patient).

In some variations, the method may include overlaying the transformed intraoral surface scan image with a volumetric scan image associated with the volumetric scan data. The overlaid images may be displayed, such as to a user. Furthermore, the method for orthodontic treatment planning may include generating an orthodontic treatment plan using the aligned intraoral surface scan data and volumetric scan data. For example, the orthodontic treatment plan may include defining a plurality of aligner trays with tooth-receiving cavities, where each aligner tray corresponds to a respective tooth arrangement.

Generally, in some variations, a system for performing automatic registration of dental image data may include one or more processors configured to receive three-dimensional intraoral surface scan data of a dentition of a patient, receive three-dimensional volumetric scan data of the dentition, generate a first set of descriptors characterizing tooth crown surfaces of the dentition from the intraoral surface scan data, generate a second set of descriptors characterizing tooth crown surfaces of the dentition from the volumetric scan data, determine a best-fit registration transform based on the first and second sets of descriptors, and align the intraoral surface scan data and the volumetric scan data based on the best-fit registration transform. In some variations, the intraoral surface scan data may include optical color scan data and the volumetric scan data may include CBCT X-ray scan data.

In some variations, at least one of the first set of descriptors and the second set of descriptors may include one or more descriptors characterizing curvature of the tooth crown surfaces. For example, such one or more descriptors may include an estimate of a local surface normal direction, a principal curvature direction, a principal curvature value, or any combination thereof, at each of the plurality of points of the tooth crown surfaces. In some variations, a descriptor may include an estimate of a local surface normal direction, two principal curvature directions, two principal curvature values associated with the two principal curvature directions, at each of the plurality of points on the tooth crown surfaces.

In some variations, one or more processors may determine the best-fit registration transform by generating one or more registration transform candidates based on matching descriptors from the first and second sets of descriptors. One or more of the registration transform candidates may be generated based on a voting scheme such as a Hough transform. Furthermore, the one or more processors may determine the best-fit registration transform by generating one or more refined registration candidates by applying an iterative local registration procedure to the one or more registration transform candidates. Various suitable techniques to generate the refined registration candidate(s) may be used, including, for example, an iterative closest point to plane algorithm or an iterative closest point to point algorithm. Additionally or alternatively, in some variations, determining the best-fit registration transform may include generating a surface proximity measure associated with each of the one or more refined registration candidates, and identifying the refined registration transform candidate associated with a lowest surface proximity measure. In some variations, the best-fit registration transform may be determined by generating a surface proximity measure associated with each of the registration transform candidates (e.g., not the refined registration candidates generated from an iterative local registration procedure).

In some variations, the intraoral surface scan data and the volumetric scan data may be aligned based on the best-fit registration transform. For example, one or more processors may align the intraoral surface scan data and the volumetric scan data by applying the best-fit registration transform to the intraoral surface scan data to generate a transformed intraoral surface scan image (e.g., by applying the best-fit registration transform to a triangulated surface of the intraoral surface scan data).

Furthermore, one or more processors may be configured to determine and apply separate best-fit registration transforms for separate portions of scan data. For example, one or more processors may determine one best-fit registration transform for an upper jaw of the patient (where this best-fit registration transform is configured to transform intraoral surface scan data of dentition on the upper jaw of the patient) and determine another best-fit registration transform for a lower jaw of the patient (where this best-fit registration transform is configured to transform intraoral surface scan data of dentition on the lower jaw of the patient).

In some variations, one or more processors may be configured to overlay the transformed intraoral surface scan images with a volumetric scan image associated with the volumetric scan data. The overlaid images may be displayed, such as to a user. Furthermore, one or more processors may be further configured to generate an orthodontic treatment plan using the aligned intraoral surface scan data and volumetric scan data. For example, the orthodontic treatment plan may include defining a plurality of aligner trays with tooth-receiving cavities, where each aligner tray corresponds to a respective tooth arrangement.

### BRIEF DESCRIPTION OF THE DRAWINGS

FIG. 1 is a schematic illustration depicting an exemplary variation of a method for automatic registration of intraoral surface and volumetric scan data.

FIG. 2 is a schematic illustration depicting an exemplary variation of a system for automatic registration of intraoral surface and volumetric scan data.

FIG. 3A depicts exemplary raw surface scan images based on intraoral surface scan data of a patient's dentition.

FIG. 3B depicts an exemplary prepared surface scan model based on the raw surface scan images depicted in FIG. 3A.

FIG. 3C depicts an exemplary segmented intraoral surface scan model of a patient's dentition.

FIG. 3D depicts a segmented and capped intraoral surface scan model of a patient's dentition.

FIG. 4A is a schematic illustration of first and second sets of descriptors for tooth crown surfaces in intraoral surface and volumetric scan data.

FIG. 4B is a schematic illustration of exemplary descriptor parameters.

FIG. 5 is a schematic illustration depicting an exemplary variation of determining a best-fit registration transform in a method for automatic registration of intraoral surface and volumetric scan data.

FIG. 6 depicts an exemplary intraoral surface scan image and an exemplary volumetric scan image that are not aligned.

FIG. 7 depicts an exemplary segmented and capped intraoral surface scan image and an exemplary volumetric scan image that are not aligned.

FIG. 8 depicts an overlay of an exemplary volumetric scan image and an exemplary intraoral surface scan image of upper dentition that has been transformed in accordance with a registration transform candidate.

FIG. 9 depicts an overlay of an exemplary volumetric scan image and exemplary transformed intraoral surface scan images of upper and lower dentition that have been transformed in accordance with the best-fit refined registration transform candidate.

### DETAILED DESCRIPTION

Non-limiting examples of various aspects and variations of the invention are described herein and illustrated in the accompanying drawings.

Described herein are methods and systems for automatic registration of image scan data for orthodontic treatment planning. For example, the methods and systems described herein may be used to accurately and precisely define a patient's anatomy (including, e.g., tooth crown, tooth root, and jawbones) at the outset of treatment, in an objective manner that is independent of inter-operator variability. Specifically, the methods and systems described herein may perform automatic registration and/or overlay of an intraoral surface scan image and a volumetric scan image (e.g., from a computed tomography scan) to define patient anatomy in an accurate and precise manner, which may provide a foundational model for further orthodontic treatment planning that is more likely to lead to a successful patient outcome and shorter treatment times.

### Methods for Automatic Registration of Image Scan Data

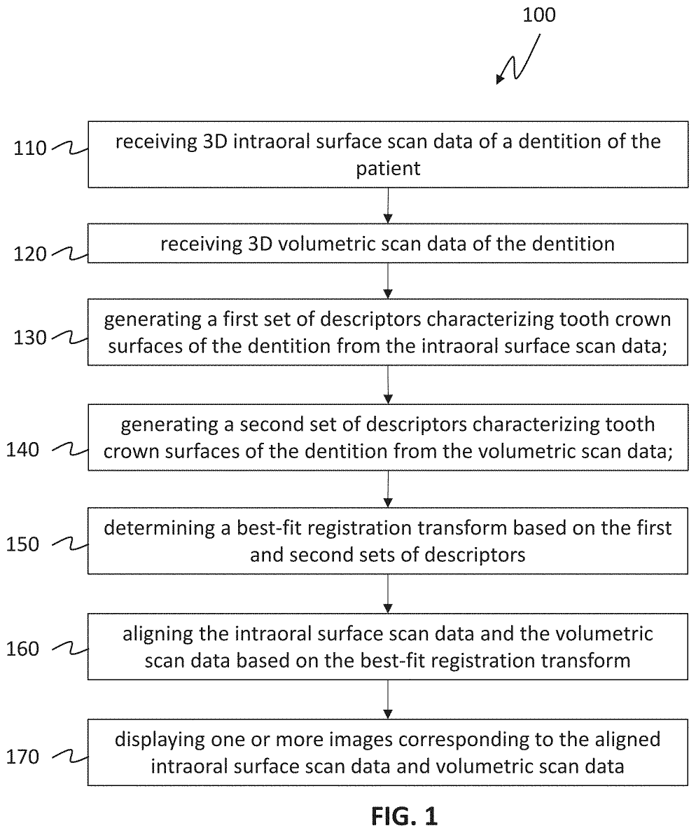

Generally, as shown in FIG. 1, in some variations, a method 100 for automatic registration of image scan data for orthodontic treatment planning may include receiving three-dimensional intraoral surface scan data of a dentition of a patient 110, receiving three-dimensional volumetric scan data of the dentition 120, generating a first set of descriptors characterizing tooth crown surfaces of the dentition from the intraoral surface scan data 130, generating a second set of descriptors characterizing tooth crown surfaces of the dentition from the volumetric scan data 140, determining a best-fit registration transform based on the first and second sets of descriptors 150, and aligning the intraoral surface scan data and the volumetric scan data based on the best-fit registration transform 160. In some variations, the method may further include displaying one or more images corresponding to the aligned intraoral surface scan data and volumetric scan data 170 (e.g., displaying an overlay of an intraoral surface scan image and a volumetric scan image, where the intraoral surface scan image and/or the volumetric scan image has been transformed such that the tooth crown surfaces in both images are aligned and/or appear in the same coordinate system).

### Receiving Scan Data

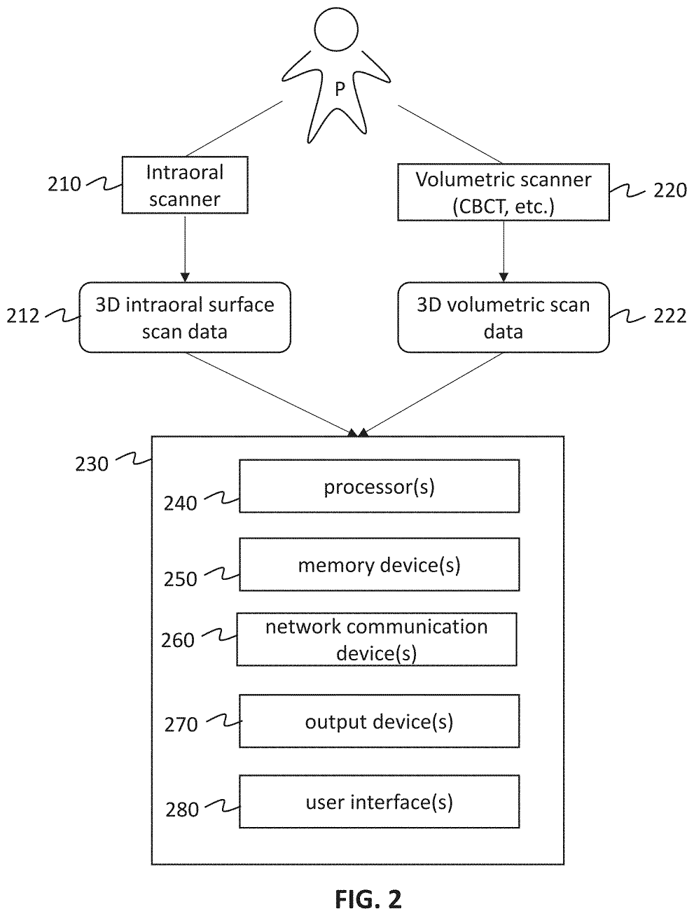

As shown in the schematic of FIG. 2, a first scan data set (e.g., intraoral surface scan data 212) and a second scan data set (e.g., volumetric scan data 222) may be generated by one or more scanning devices configured to obtain anatomical imaging data for a patient P. For example, an intraoral scanning device 210 may be used by a practitioner or other user to obtain image data (e.g., optical color scan data) representative of external surfaces of a patient's dentition (e.g., teeth crowns, gingiva, etc.). The intraoral scanning device may, for example, be a handheld scanner that emits light toward the patient's dentition as the scanner is manipulated inside the mouth of the patient. The emitted light reflects off surfaces of the patient's dentition, and the reflected light is captured by the intraoral scanner and subsequently analyzed to transform the reflected light data into surface imaging data. An exemplary intraoral scanner suitable for use in obtaining three-dimensional intraoral surface scan data 212 is the CS 3600 intraoral scanner available from CARESTREAM DENTAL LLC (Atlanta, Ga., USA). However, any suitable intraoral scanners may be used to obtain such intraoral surface scan data 212.

Generally, the digitized surfaces of the patient's dentition obtained from the intraoral surface scan may be used to create one or more patient-customized orthodontic appliances (e.g., using computer-aided design and computer-aided manufacturing (CAD/CAM) technology), which may, for example, be used to apply forces to teeth and induce controlled orthodontic tooth movement (OTM). Accurate surface scan data of a patient's teeth enable such appliances to have a predictably intimate fit to the unique curvatures of the teeth. Moreover, precise manipulation of accurate intraoral surface scan data allows the creation of orthodontic appliances to induce effective OTM.

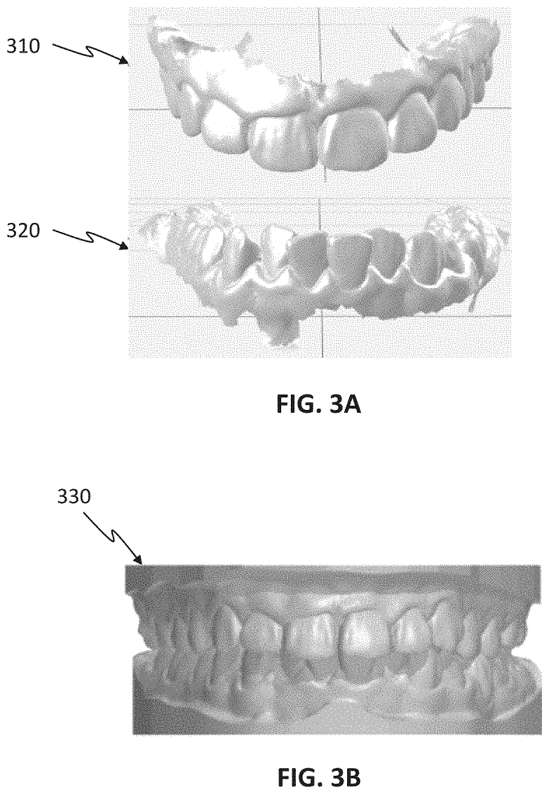

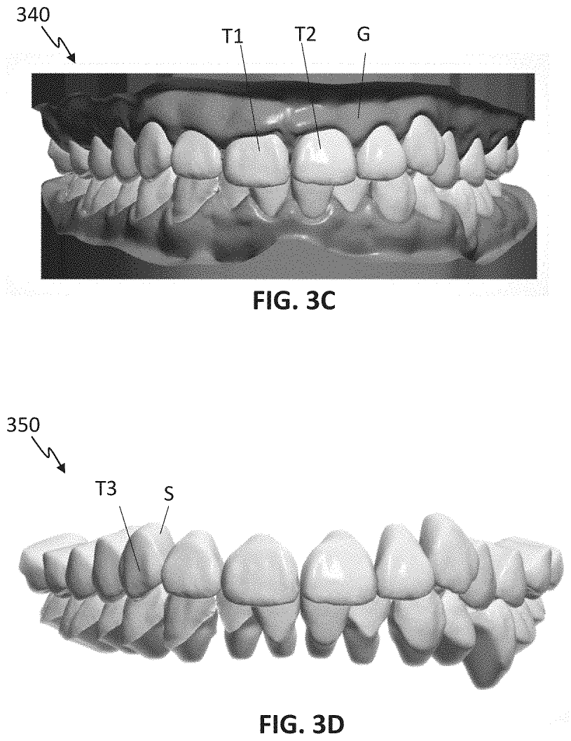

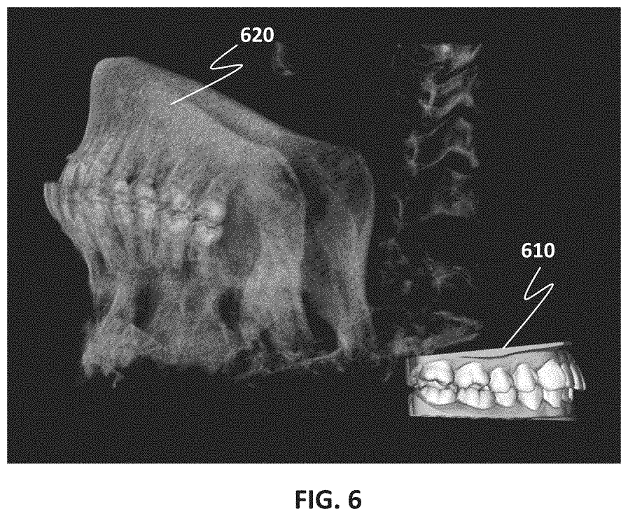

FIG. 3A illustrates an exemplary raw surface scan image based on intraoral surface scan data for a patient's upper dentition 310 and lower dentition 320. As shown in FIG. 3A, while intraoral surface scan data may provide information about an external form of the tooth crowns and at least a portion of gingiva, the intraoral surface scan data does not supply direct information about certain other tooth structures such as a length and a size of the underlying tooth roots and bone. In some variations, the raw surface scan data may be manipulated into a prepared (e.g., processed) surface scan model such as the model 330 shown in FIG. 3B, or the model 610 shown in FIG. 6. For example, the scan images may be trimmed to include only relevant scan data, and/or placed onto a template jaw base model, etc.

In some variations, the volumetric scan data 222 may be obtained by a volumetric scanner 220. In some variations, the volumetric scanner may be configured to provide three-dimensional X-ray imaging (e.g., cone-beam computed tomography (CBCT)) of dentition (e.g., crowns, gingiva, root structures) and craniofacial features (e.g., bone). The volumetric scanner may also be configured to provide detailed information regarding each tooth's root orientation. An exemplary CBCT X-ray scanner suitable for use in obtaining three-dimensional volumetric scan data 222 is the RAYSCAN α imaging device available from RAY COMPANY (RAY AMERICA, Inc., Fort Lee, N.J., USA). However, any suitable extraoral scanners providing volumetric information of dentition and craniofacial features may be used to obtain the volumetric scan data 222.

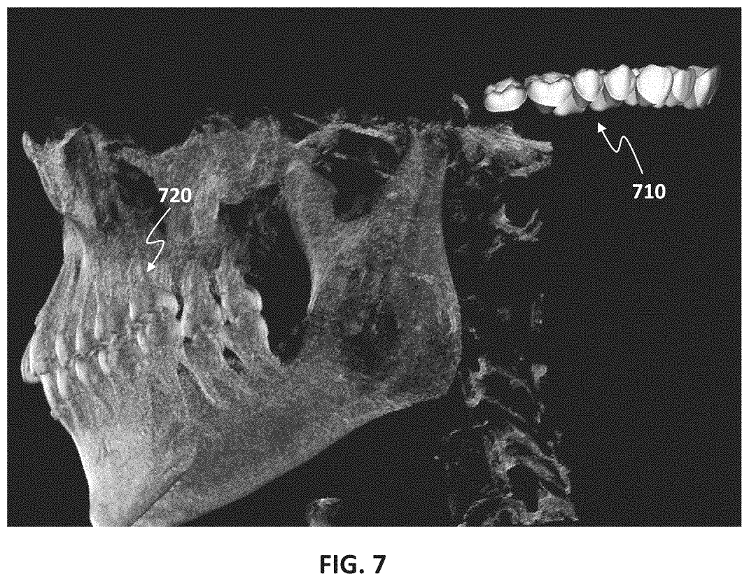

Generally, the volumetric scan data obtained from an ionizing or non-ionizing volumetric scanner may be used to identify patient anatomical features such as bone structures, crowns and roots of teeth, and/or pathology of the craniofacial region, as well as to measure or otherwise quantify other patient characteristics such as airway volume, facial phenotype, and/or malocclusion of the jaws. For example, as shown in FIG. 6 depicting an exemplary volumetric scan image 620 and FIG. 7 depicting an exemplary volumetric scan image 720, the volumetric scan data may provide one or more of the volume, length, and/or morphology of tooth roots and/or craniofacial features. The information relating to tooth roots may improve the modeling of orthodontic tooth movement by, for example, allowing the specification of each tooth's actual center of resistance and long axis for rotation within the patient's jawbone, as further described in U.S. Pat. No. 10,905,526, which is incorporated herein in its entirety by this reference.

### Model Segmenting

In some variations, discrete portions of a model corresponding to the surface scan data and/or a model corresponding to the volumetric scan data may be identified in a model segmentation process. For example, different portions of a model, where the different portions correspond to different anatomical features, may be segmented. For example, different teeth in the model may be segmented in order to enable independent selection, viewing, and/or manipulation of each tooth in isolation. In some variations, at least the root structure of at least one tooth in the model may be separated (as a discrete, identifiable volume) from the rest of the model. Additionally or alternatively, the model may be segmented to separate other anatomical features such as the crown of each tooth, gingiva, periodontal ligament(s), and bone.

For example, FIG. 3C depicts a segmented model 340 corresponding to intraoral surface scan data, in which individual teeth (e.g., T1 and T2) are segmented (e.g., identified) as separate structures, and the teeth are further segmented with respect to gingiva (G). In some variations, model segmentation may further include isolating one or more anatomical features, such as to enable individual selection and display of an isolated feature, for example. For example, FIG. 3D depicts a portion of an exemplary intraoral surface scan model 350 including tooth crowns that are segmented and “capped” with a smooth surface (e.g., tooth T3 is segmented and capped with a surface S). FIG. 7 depicts another example of an intraoral surface scan model 710 including tooth crowns that are segmented and “capped” with a smooth surface. Similarly, other portions of the intraoral surface scan model (gingiva, tooth roots, etc.) and/or volumetric scan model may be segmented and capped for display and/or analysis. In some variations, the model corresponding to the surface scan data and the model corresponding to the volumetric scan data may each be individually and separately segmented and/or capped. Alternatively, in some variations, segmentation and/or capping may alternatively be performed on an integrated patient model obtained by previously overlaying the surface scan data and the volumetric scan data as described below.

In some variations, model segmentation may be performed with manual input. For example, similar to that described above, markers may be placed on the model by a user through the user interface to designate one or more anatomical features for defining segmentation boundaries, such as a plane or other surface between two teeth, between a tooth and gingiva, or between a tooth crown and a tooth root. Alternatively, markers may be placed on the integrated patient model after overlaying the scan data in the overlaying process described below. Once placed, the markers may be adjusted by the user. In other variations, markers denoting segmentation boundaries may be automatically suggested and placed by a software algorithm (and may be confirmed and/or adjusted based on user manual input). For example, proposed segmentation boundaries may be automatically defined based on color pixel data in the surface scan data and/or overlaid integrated patient model. As an illustrative example, a threshold color channel intensity change between adjacent pixels in the surface scan data and/or overlaid integrated patient model may indicate a transition between a light-colored tooth and a darker-colored or pink gingiva.

Furthermore, in some variations, model segmentation may be automatically performed based at least in part on voxel density of various voxels in the volumetric scan model (and/or integrated patient model). Different kinds of patient tissue will be represented with different voxel density in the volumetric scan data, as the result of the differing radiopacity of different kinds of tissue. For example, bones have relatively higher radiopacity than gingiva, and therefore will be represented with greater voxel density than gingiva in a CBCT scan. As another example, tooth enamel and root dentin have a higher radiopacity than their surrounding alveolar bone, and will be represented with greater voxel density than surrounding bone in the volumetric scan data. Accordingly, in some variations, different regions in the integrated patient model may be automatically identifiable by monitoring threshold changes in voxel density across neighboring voxels in the integrated patient model, thereby aiding segmentation.

In some variations, partial or full segmentation of both the surface scan model and the volumetric scan model may be performed prior to registering the models to form an integrated patient model. In some variations, partial or full segmentation of the integrated patient model may be performed after registering the (at least partially) unsegmented surface scan and volumetric scan models. In yet other variations, either the surface scan model or the volume scan model may be segmented after their overlay, based at least in part on alignment information derived from the integrated patient model. For example, a pre-segmented intraoral surface scan model may have data about tooth-tooth boundaries and/or tooth-gingiva boundaries, and may be utilized to “seed” or otherwise inform the identification and segmentation of the aligned volumetric data for the interface between the tooth root and bone. For example, the gingival margin of a tooth may be extrapolated toward the tooth's root apex, in that the density value of the voxels around the tooth margin identified in the intraoral surface scan may seed the identification and growth of that tooth's associated root along the root-bone boundary.

### Estimating Locations of Tooth Crown Surfaces

In some variations, the general locations of tooth crown surfaces may be estimated in the scan data prior to generating descriptors of the tooth crown surfaces. In some variations, the locations of the tooth crown surfaces are easily identified in the segmented intraoral surface scan model (or otherwise are inherent in the raw surface scan data).

Additionally or alternatively, in some variations, the tooth crown surfaces corresponding to the volumetric scan data can be distinguished from other anatomical structures (e.g., gingiva, tooth root) based at least in part on bone tissue density, which may correspond to volumetric pixel intensity in the volumetric scan data. For example, tissue in a particular region in the volumetric scan data may be automatically identified as bone tissue if volumetric pixel intensity for that region is above a predetermined threshold. In an exemplary variation, tissue in the volumetric scan data may be identified as bone tissue where the tissue has a density within the 2.5% highest density quantile threshold for the given scan. However, other suitable thresholds may be used. In some variations, types of tissue in the volumetric scan data may be additionally or alternatively distinguished based on anticipated or expected radiodensity for different tissue types. For example, tissue regions in the volumetric scan data having a Hounsfield unit (HU) value of about 2050 (or within a predetermined margin of this value, such as within 5% or within 10% of this value) may be considered enamel (e.g., tooth crown). Tissue regions having a HU value of about 1310 (or within a predetermined margin of this value, such as within 5% or within 10% of this value) may be considered dentine, tissue regions having a HU value of about 705 (within a predetermined margin of this value, such as within 5% or within 10% of this value) may be considered pulp, and tissue regions having a HU value of about 190 or within a predetermined margin of this value, such as within 5% or within 10% of this value) may be considered maxillary spongy bone (alveolar) tissue. Furthermore, tooth crown surfaces and other bone tissue (e.g., jawbones) among the identified bone tissue in the volumetric scan data may additionally or alternatively be automatically distinguished based on general spatial location and/or shape of the identified bone tissue (e.g., based on expected general region or appearance of dentition and/or jawbones, etc.).

Additionally or alternatively, general locations of tooth crown surfaces may be indicated manually in imaging software, such as by a user (e.g., doctor or other clinician, technician, orthodontic device manufacturer) applying a set of virtual markers in the intraoral surface scan model and/or volumetric scan model to indicate boundary and/or interior of regions of the models that includes tooth crown surfaces. In some variations, tentative or initial general locations of tooth crown surfaces in the intraoral surface scan data and/or volumetric scan data may be determined automatically, and then manually confirmed and/or adjusted as appropriate by a user.

### Generating Descriptors

As shown in FIG. 1, the method 100 may include generating a first set of descriptors characterizing tooth crown surfaces of the dentition from the intraoral surface scan data 130, and generating a second set of descriptors characterizing tooth crown surfaces of the dentition from the volumetric scan data 140. Generally, the descriptors function to quantify one or more curvature features of the tooth crown surfaces in the patient's dentition.

In some variations, the first set of descriptors and/or the second set of descriptors may be generated once the general locations of tooth crown surfaces in the scan data are determined. Each descriptor characterizes one or more curvature features of the tooth crown surfaces at a respective point (location) on the tooth crown surfaces. Furthermore, each set of descriptors may include descriptors for a suitable number of points on the tooth crown surfaces to characterize the overall geometry of the tooth crown surfaces as represented in the intraoral surface scan data or the volumetric scan data.

In some variations, descriptors may be generated for a set of random points on the tooth crown surfaces, or descriptors may be generated at predetermined set of points on the tooth crown surfaces (e.g., in a grid pattern, such as in a triangular grid or rectangular grid). Additionally or alternatively, in some variations more descriptors may be generated in general regions on the tooth crown surfaces that may be anticipated to have more variance in curvature (e.g., masticatory surfaces of molars).

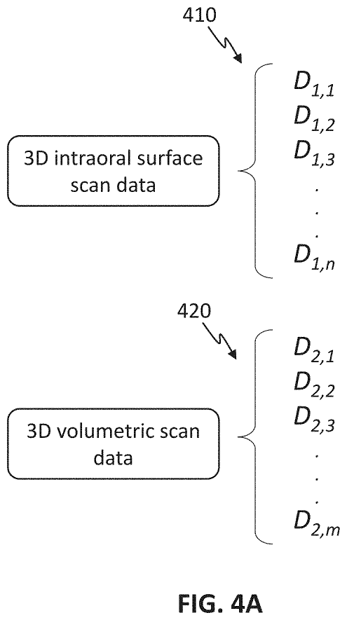

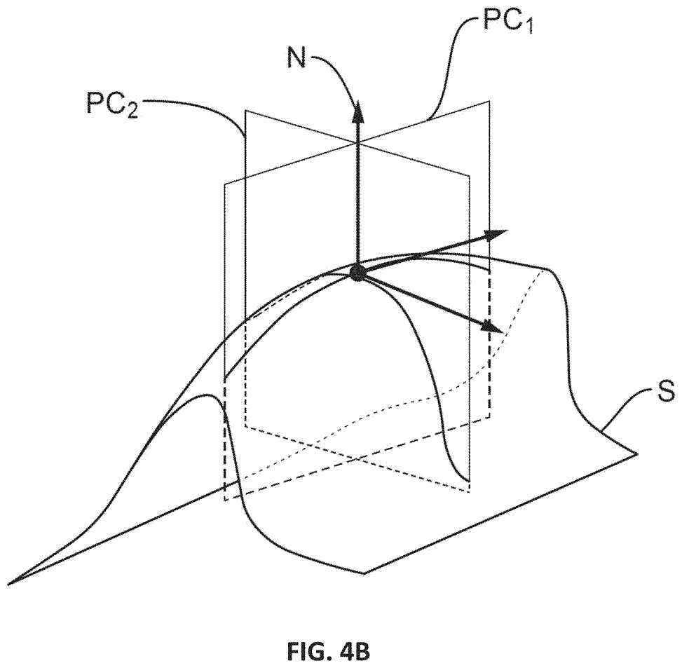

Any suitable number of descriptors may be generated for any suitable number of points on each scan. As shown in FIG. 4A, the first set of descriptors D_{1,1} through D_{1,n} may include a first number of descriptors (n descriptors) for a first number of points (n points) on the tooth crown surfaces identified in the intraoral scan data. Similarly, the second set of descriptors D_{2,1} through D_{2,m} may include a second number of descriptors (m descriptors) for a second number of points (m points) on the tooth crown surfaces identified in the volumetric scan data. In some variations, the first and second sets of descriptors may include descriptors for the same number of points for the intraoral surface scan data and the volumetric scan data (n=m), while in other variations the first and second sets may include descriptors for different numbers of points for the intraoral surface scan data and the volumetric scan data (n≠m). For example, in some variations the second set of descriptors for the volumetric scan data may include more descriptors than the first set of descriptors for the intraoral scan data, or vice versa. In some variations, the second set of descriptors for the volumetric metric scan data may include more descriptors than the first set of descriptors for the intraoral scan data to account for the estimated volumetric tooth crown surface typically including many unrelated patches of bone and root surface. For example, in some variations, the ratio of descriptors for the volumetric scan data to descriptors for the intraoral surface scan may be at least 3:1, at least 4:1, at least 5:1, at least 7:1, at least 10:1, or at least 15:1.

In an exemplary variation, about 50,000 descriptors may be generated at a set of random surface points for the estimated tooth crown surface in the volumetric scan data, and about 300 descriptors may be generated at a set of random surface points for every tooth (e.g., segmented tooth) in the intraoral scan data. In some variations, the descriptors for all teeth in the intraoral scan data may be merged into one set, such that the first set of descriptors for the intraoral scan data may include up to about 4,800 descriptors per upper or lower jaw, for example. However, it should be understood that other suitable numbers of descriptors for the volumetric scan data and the intraoral scan data may be generated, such as for a desired resolution. For example, for the volumetric scan data at least 10,000 descriptors, at least 15,000 descriptors, at least 20,000 descriptors, at least 25,000 descriptors, at least 30,000 descriptors, at least 40,00 descriptors, at least 50,000 descriptors, at least 75,000 descriptors, or at least 100,000 may be generated. As another example, for the intraoral surface scan data at least 100 descriptors, at least 200 descriptors, at least 300 descriptors, at least 400 descriptors, or at least 500 descriptors may be generated per tooth.

As described above, each descriptor may include one or more suitable parameters to characterize the curvature of the tooth crown surfaces at a particular point. For example, in some variations, a descriptor may include an estimation of a local surface normal, one or more principal curvature directions, and/or one or more principal curvature values (e.g., curvature radius values) at a particular point. As shown in FIG. 4B, the local surface normal (N) is a vector that is perpendicular to the tangent plane at a given point on the surface (e.g., tooth crown surface). The principal curvature directions and principal curvature values specify the principal curvatures (PC₁ and PC₂) at a given point; that is, the directions and values of minimum curvature and maximum curvature at a given point. The principal curvature directions (PC₁ and PC₂) are orthogonal to the surface normal (N).

In an exemplary variation, a descriptor for a given tooth crown surface may include a local surface normal for that point, a direction and value of a first principal curvature for that point, and a direction and value of a second principal curvature for that point. In this variation, a descriptor D for a given point p may include 3D coordinates (e.g., Cartesian coordinates, spherical coordinates) for the given point p, and a curvature tensor C having three rows describing curvature of the tooth crown surface at point p. As shown in Equation 1 below, the first row may represent the local surface normal n, the second row may represent a first principal curvature c₁, and the third row may represent a second principal curvature c₂ for the given point p:

$$D = \{ C = ( \begin{matrix}\vec{n} \\ \vec{{c}_{1}} \\ \vec{{c}_{2}}\end{matrix} ) , \vec{p} \} \qquad eqn . ( 1 )$$

The curvature tensor C may be determined using any one or more suitable techniques. For example, the principal curvatures may be determined at least in part by taking point samples around the given point p to calculate a local estimation of the second fundamental form matrix and diagonalizing this matrix. The principal curvature directions may be obtained as the eigenvectors of the diagonalized matrix, and the principal curvature values may be obtained as the eigenvalues of the diagonalized matrix. An exemplary technique to estimate the curvature tensor C is described in further detail by Taubin (Taubin, Gabriel, “Estimating the tensor of curvature of a surface from a polyhedral approximation”, Proceedings of IEEE International Conference on Computer Vision, pp. 902-907 (1995), which is incorporated herein by reference).

In some variations, the first and/or second sets of descriptors may be filtered, so as to isolate meaningful curvature information among the descriptors. For example, the first and/or second sets of descriptors may be filtered to remove noise. Additionally or alternatively, the first and/or second sets of descriptors may be filtered to remove descriptors that represent curvature information considered to be unreasonable. For example, in some variations, descriptors with principal curvature values that are outside a predetermined (e.g., reasonable) tooth surface curvature range (or below or above a predetermined curvature radius value) may be removed. As an illustrative example, descriptors with principal curvature radius values that are below about 0.4 mm may be filtered out, such that descriptors with principal curvature radius values at or above about 0.4 mm may be retained for further analysis. However, other curvature radius threshold(s) may be used to filter the first and/or second sets of descriptors. For example, in some variations, descriptors with principal curvature radius values that are below a lower threshold value of between about 0.1 mm to about 0.8 mm may be filtered out (i.e., such that descriptors with principal curvature radius values at or above about 0.1 mm, or at or above 0.2 mm, or at or above 0.3 mm, etc. may be retained for further analysis).

### Determining Registration Transform(s)

As shown in FIG. 1, the method 100 may include determining a best-fit registration transform based on the first and second sets of descriptors 150 (e.g., the filtered sets of descriptors). The best-fit registration transform may function to transform at least one of the intraoral surface scan data and the volumetric scan data such that the intraoral surface scan image and the volumetric scan image are aligned and/or appear in the same coordinate system. For example, FIG. 7 depicts an exemplary intraoral surface scan image 710 and volumetric scan image 720, where the two images are not aligned with one another. However, through a series of techniques, a best-fit registration transform may transform the scan data for one or both images such that the images are aligned, as shown in FIG. 10. Furthermore, in some variations, the method may include determining separate best-fit registration transforms for upper dentition and lower dentition (e.g., two best-fit registration transforms may be determined).

Generally, in some variations, the best-fit registration transform may be identified from multiple initial registration transform candidates generated based on “matching” descriptors from the first and second sets of descriptors that have similar curvature values. In some variations, a set of one or more refined, or fine-tuned, registration transform candidates may be derived from the initial set of multiple registration transform candidates. A best-fit registration transform may be selected from any of the registration transform candidates (e.g., from the refined registration transform candidates, or directly from the initial registration transform candidates), as further described below.

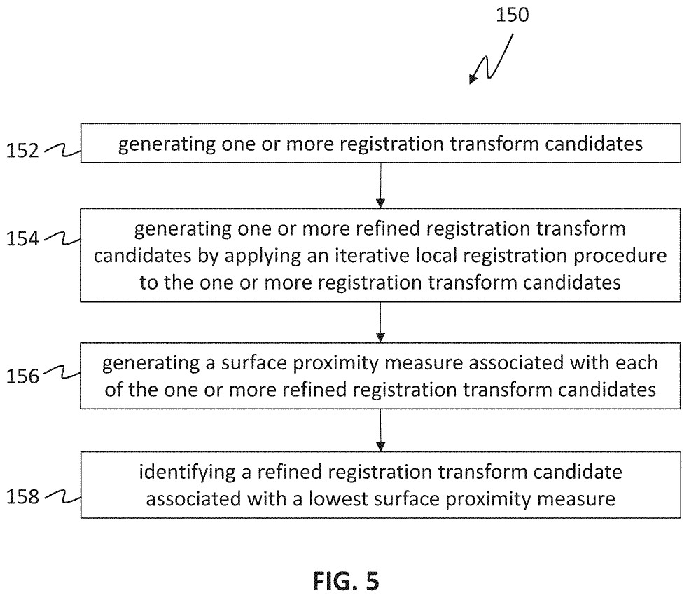

For example, as shown in FIG. 5, in determining the best-fit registration transform 150, the method 100 may include generating one or more registration transform candidates 152 based on the first and second sets of descriptors, generating one or more refined registration transform candidates 154 by applying an iterative local registration procedure to the one or more registration transform candidates, generating a surface proximity measure associated with each of the one or more refined registration transform candidates 156, and identifying a refined registration transform candidate associated with a lowest surface proximity measure 158. In some variations, the refined registration transform candidate associated with a lowest surface proximity measure may be identified as the best-fit registration transform candidate.

In some variations, generating one or more registration transform candidates 152 may include generating registration transform candidate(s) based on matching descriptors having similar curvature values, as described above. By solving a system of linear equations, a procedure for matching two descriptors with similar principal curvature values can be used to determine the relative transformation from one descriptor to the other, so that the corresponding principal directions and normals in the two descriptors are aligned.

For example, similar to Equation 1 described above, a pair of two descriptors D₁ and D₂ for given points p₁ and p₂, respectively, may be expressed below as Equations 2 and 3:

$${D}_{1} = \{ {C}_{1} = ( \begin{matrix}{\vec{n}}_{1} \\ \vec{{c}_{11}} \\ \vec{{c}_{12}}\end{matrix} ) , \vec{{p}_{1}} \} \qquad eqn . ( 2 )$$

$${D}_{2} = \{ {C}_{2} = ( \begin{matrix}{\vec{n}}_{2} \\ \vec{{c}_{21}} \\ \vec{{c}_{22}}\end{matrix} ) , \vec{{p}_{2}} \} \qquad eqn . ( 3 )$$

For example, descriptor D₁ may be from the first set of descriptors characterizing tooth crown surfaces in the intraoral surface scan data, and descriptor D₂ may be from the second set of descriptors characterizing tooth crown surfaces in the volumetric scan data. Given this pair of descriptors D₁ and D₂, the goal is to identify a transform T including a 3D rotation matrix R and a shift vectors to align p₁ with p₂ and to align C₁ with C₂. The transform T may be determined by solving a 3×3 linear equation system of Equations (4)-(6) as shown below, where the inverse of matric C is equal to its transpose because the matrix is orthogonal:

$$T = \{ R , \vec{s} \} : \{ \begin{matrix}{RC}_{1} = {C}_{2} \\ R \vec{{p}_{1}} + \vec{s} = \vec{{p}_{2}}\end{matrix} \} \qquad eqn . ( 4 )$$

$$R = {C}_{2} {C}_{1}^{- 1} = {C}_{2} {C}_{1}^{T} \qquad eqn . ( 5 )$$

$$\vec{s} = \vec{{p}_{2}} - R \vec{{p}_{1}} \qquad eqn . ( 6 )$$

By solving the above-described system of linear equations for matching descriptors for the first set of descriptors (associated with the intraoral surface scan data) and the second set of descriptors (associated with the volumetric scan data), a set of initial registration transform candidates T may be generated. In some variations, a voting scheme such as a generalized Hough transform may be used to obtain a list of approximate registration transform candidates. The generalized Hough transform provides a voting scheme in the space of all possible transformations to find approximate transforms with the most “votes”, where each vote represents a pair of matching descriptors from the two sets.

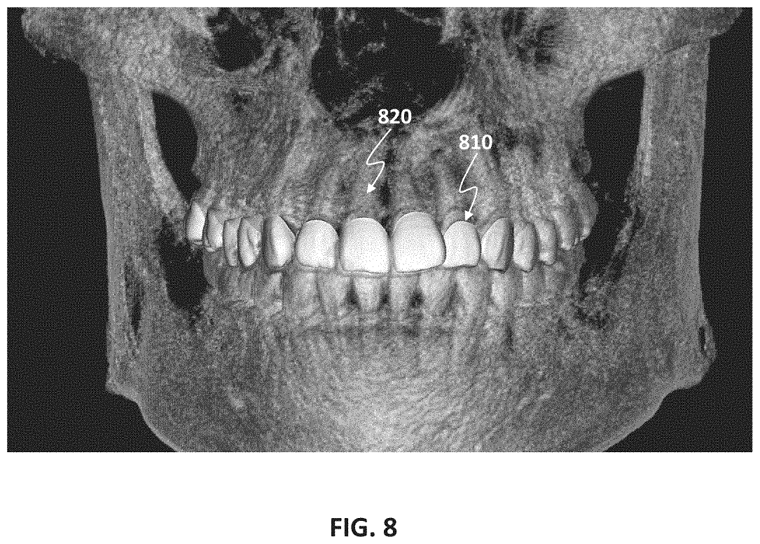

FIG. 8 illustrates an exemplary overlay of a volumetric scan image 820 and a transformed intraoral surface scan image 810 of upper dentition that has been transformed (e.g., rotated and/or shifted) in accordance with a registration transform candidate obtained by the voting scheme. As shown in FIG. 8, the volumetric scan image 820 and transformed intraoral surface scan image 810 are in close alignment but not perfect because the registration transform candidate has not yet been refined as described below. A separate registration transform candidate may be similarly identified for the lower dentition and be used to transform the intraoral surface scan image of lower dentition in a similar manner (not shown), to be aligned with the volumetric scan image 820.

Using the initial registration transform candidates, the method may further include generating one or more refined registration transform candidates 154 as described above. In some variations, generating one or more refined registration transform candidates may include applying an iterative local registration procedure to the one or more initial registration transform candidates. For example, in some variations, the method may include applying an iterative closest point-to-plane algorithm, such as that described by Low (Low, Kok-Lim, “Linear Least-Squares Optimization for Point-to-Plane ICP Surface Registration”, Technical Report TR04-004, Department of Computer Sciences, University of North Carolina at Chapel Hill (2004), which is incorporated herein by reference). However, in some variations, other suitable local registration procedures may be performed to generate one or more refined registration transform candidates, such as an iterative closest point-to-point algorithm.

In some variations, a best-fit registration transform may be identified from the refined registration transform candidate((s). In some variations, determining the best-fit registration transform may include generating a surface proximity measure associated with each of the one or more refined registration transform candidates. For example, to generate a surface proximity measure for a given registration transform candidate that transforms the intraoral scan data, for each point sampled in the transformed intraoral scan surface, the closest point with a similarly-directed normal may be found on the volumetric scan surface (e.g., where the dot product of the two normal is greater than a predetermined threshold, such as 0.7). Generally, the surface proximity measure for a particular registration transform candidate may be calculated as the sum of squared distances between such points. However, in some variations, for a particular point on the transformed intraoral scan surface, if the distance to the closest point on the volumetric scan surface is higher than a predetermined threshold value, then the squared threshold value (instead of the actual squared distance value) is added to the sum for that particular point's contribution toward the surface proximity measure. In some variations, the threshold values may be for example, between about 0.1 mm and about 0.5 mm. In an exemplary variation, the threshold value may be about 0.2 mm.

As another example, instead of identifying the closest point with solely a similarly-directed normal when determining the surface proximity measure, generating a surface proximity measure may involve identifying the closest point on the volumetric scan surface that has both a similarly-directed normal and similarly-directed principal curvature directions as the point under consideration in the transformed intraoral scan surface. As yet another example, the method may include generating a surface proximity measure that disregards any requirement of sufficiently similarly-directed normal direction or sufficiently similarly-directed principal curvature directions when looking for the closest point (i.e., in this example, the closest point on the volumetric scan surface need not have a similarly-directed normal, nor similarly-directed curvature directions, compared to the point in the intraoral scan surface under consideration).

Although the methods described herein are primarily described as using a surface proximity measure to identify the best-fit registration transform among the refined registration transform candidates, it should be understood that a surface proximity measure may be used to identify a best-fit registration transform from initial registration transform candidates. For example, such as in instances where refined registration transform candidates are not generated, the method may include identifying a best-fit registration candidate from the initial registration transform candidates, such as using the same surface proximity measure calculations as described above.

### Aligning Scan Data

After a best-fit registration transform is determined, the method may include aligning the intraoral surface scan data and the volumetric scan data based on the best-fit registration transform 160. For example, to align the scan data, the transform may be applied to a triangulated surface of the intraoral scan image based on the intraoral scan data. As described above, in some variations separate registration transforms may be determined for upper dentition and lower dentition. As such, the intraoral surface scan data for the upper dentition may be aligned to the volumetric scan data based on a first best-fit registration transform, and the intraoral surface scan data for the lower dentition may be aligned to the volumetric scan data based on a second best-fit registration transform. Once the scan data are aligned, the transformed intraoral scan image(s) may be overlaid or merged with the volumetric scan image for display.

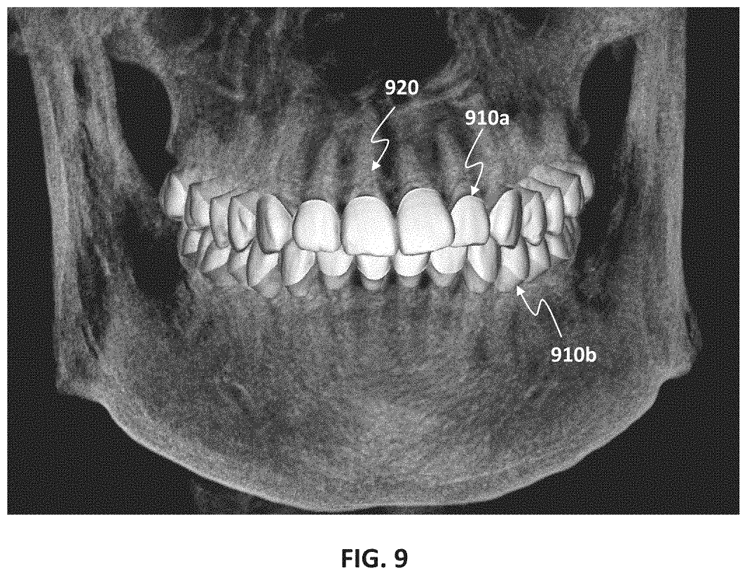

FIG. 9 illustrates an exemplary overlay of a volumetric scan image 920 and transformed intraoral surface scan images 910a and 910b that have been transformed (e.g., rotated and/or shifted) in accordance with respective best-fit registration transforms. Specifically, transformed intraoral surface scan image 910a of upper dentition has been transformed with a first best-fit registration transform calculated for the upper dentition intraoral surface scan to be aligned with the volumetric scan image 920, and transformed intraoral surface scan image 910b of lower dentition has been transformed with a second best-fit registration transform calculated for the lower dentition intraoral surface scan to be aligned with the volumetric scan image 920. As shown in FIG. 9, the volumetric scan image 920 and the transformed intraoral surface scan images 910a and 910b are in very close alignment due to the nature of the best-fit registration transforms. The teeth of the intraoral surface scan image(s) are segmented and aligned with the crowns and roots of the volumetric scan image voxels. In some variations, some or all of the aligned images may form an integrated patient model (as a combination of aligned intraoral surface scan data and volumetric scan data).

While the methods described herein are primarily described with respect to identifying a transform that is used to transform the intraoral scan data to be aligned with the volumetric scan data, alternatively the identified transform may be used to transform the volumetric scan data to be aligned with the intraoral scan data.

### Displaying Aligned Images

In some variations, the aligned scan images may be displayed on a suitable display (e.g., computer monitor or screen) to a user. For example, the method may include displaying one or more images corresponding to the aligned intraoral surface scan data and volumetric scan data 170. The volumetric scan data 170 may be displayed, for example, transformed intraoral surface scan image(s) of upper dentition and/or transformed intraoral surface scan image(s) of lower dentition. In some variations, the transformed intraoral surface scan image of upper dentition and/or the transformed intraoral surface scan image of lower dentition may be independently and selectively displayed, such as to display one or both in response to a user input. Additionally or alternatively, in some variations, the volumetric scan image may be selectively displayed, such as in response to a user input.

Displaying the aligned scan images may enable an orthodontic treatment team (e.g., doctor or other clinician, orthodontic device manufacturer) to visualize the integrated patient model for treatment planning purposes, as described herein.

### Treatment Planning

In some variations, utilizing the automatically-registered integrated patient model (as a combination of intraoral surface scan data and volumetric scan data) enables greater accuracy and precision in characterizing patient anatomy and providing a better foundation for further treatment planning. Accordingly, in some variations, further treatment planning may be performed utilizing the integrated patient model, such as by taking into account tooth roots and the like. In some variations, the center of rotation of one or more teeth of the patient may be identified in the integrated patient model as described in U.S. Pat. No. 10,905,526, which was incorporated by reference above.

In some variations, treatment planning may include generating a series of one or more aligner trays with tooth-receiving cavities, each aligner tray corresponding to a respective tooth arrangement such that a patient wearing the series of aligner trays in a particular sequential order (e.g., one tray per one week, two weeks, three weeks, or other suitable period of time) experiences a gradual transition of their dentition from an original tooth arrangement to a desired or targeted tooth arrangement. The forms of the aligner trays may correspond to different stages that gradually move each of one or more teeth. For example, each aligner tray may to a respective tooth arrangement such that the series of aligner trays progressively move teeth in treatment paths (e.g., in accordance with their centers of rotation for natural movement, as described in U.S. Pat. No. 10,905,526). The aligner trays may, for example, be formed from rigid or semi-rigid polymer (e.g., through vacuum forming, injection molding, 3D printing, etc.). The aligner trays may be provided to a patient individually (e.g., shipped one at a time according to predetermined intervals) or in one or more sets.

### Systems for Automatic Registration of Image Scan Data

FIG. 2 illustrates various components of an exemplary system for orthodontic treatment planning. Specifically, an exemplary system may include a general computing device 230 including one or more processors 240, one or more memory devices 250, one or more network communication devices 260, one or more output devices 270, and/or one or more user interfaces 280. Exemplary general computing devices include a desktop computer, laptop computer, and mobile computing devices (e.g., tablets, mobile phones).

The processor 240 may be any suitable processing device configured to run and/or execute a set of instructions or code, and may include one or more data processors, image processors, graphics processing units, physics processing units, digital signal processors, and/or central processing units. The processor may be, for example, a general purpose processor, a Field Programmable Gate Array (FPGA), an Application Specific Integrated Circuit (ASIC), and/or the like. The processor may be configured to run and/or execute application processes and/or other modules, processes and/or functions associated with the system and/or a network associated therewith. The underlying device technologies may be provided in a variety of component types (e.g., MOSFET technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and/or the like.

In some variations, the memory 250 may include a database and may be, for example, a random access memory (RAM), a memory buffer, a hard drive, an erasable programmable read-only memory (EPROM), an electrically erasable read-only memory (EEPROM), a read-only memory (ROM), Flash memory, and the like. The memory may store instructions to cause the processor to execute modules, processes, and/or functions such as scan data processing and alignment. In some variations, the memory 250 may receive intraoral surface scan data 212 and/or volumetric scan data 222 in full (e.g., DICOM files generated by scanner-specific software). Additionally or alternatively, the memory 250 may receive intraoral surface scan data 212 and/or volumetric scan data 222 in parts, such as in a real-time or near real-time feed of data directly from the intraoral scanner 210 and/or volumetric scanner 220.

Some variations described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for the specific purpose or purposes.

Examples of non-transitory computer-readable media include, but are not limited to, magnetic storage media such as hard disks, floppy disks, and magnetic tape; optical storage media such as Compact Disc/Digital Video Discs (CD/DVDs); Compact Disc-Read Only Memories (CDROMs), and holographic devices; magneto-optical storage media such as optical disks; solid state storage devices such as a solid state drive (SSD) and a solid state hybrid drive (SSHD); carrier wave signal processing modules; and hardware devices that are specially configured to store and execute program code, such as Application-Specific Integrated Circuits (ASICs), Programmable Logic Devices (PLDs), Read-Only Memory (ROM), and Random-Access Memory (RAM) devices. Other variations described herein relate to a computer program product, which may include, for example, the instructions and/or computer code disclosed herein.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

Furthermore, one or more network communication devices 260 may be configured to connect the general computing device to another system (e.g., intraoral scanner 210, volumetric scanner 220, Internet, remote server, database, etc.) by wired or wireless connection. In some variations, the general computing device may be in communication with one or more other general computing devices via one or more wired or wireless networks. In some variations, the communication device may include a radiofrequency receiver, transmitter, and/or optical (e.g., infrared) receiver and transmitter configured to communicate with one or more device and/or networks. In an exemplary variation, the network communication devices 260 may include a cellular modem (e.g., 3G/4G cellular modem) such that it is advantageously not dependent on internet Wireless Fidelity (WiFi®) access for connectivity.

Alternatively, wireless communication may use any of a plurality of communication standards, protocols, and technologies, including but not limited to, Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth®, WiFi®, voice over Internet Protocol (VoIP), or any other suitable communication protocol. In some variations, the devices herein may directly communicate with each other without transmitting data through a network (e.g., through NFC, Bluetooth®, RFID, and the like). For example, devices (e.g., one or more computing devices, an intraoral scanner 210, and/or a volumetric scanner 220, etc.) may directly communicate with each other in pairwise connection (1:1 relationship), or in a hub-spoke or broadcasting connection (“one to many” or 1:m relationship). As another example, the devices (e.g., one or more computing devices and/or intraoral scanner 210, and/or volumetric scanner 220, etc.) may communicate with each other through mesh networking connections (e.g., “many to many”, or m:m relationships), such as through Bluetooth mesh networking.

As described above, the computing device in the system may include one or more output devices 270 such a display and/or audio device for interfacing with a user. For example, an output device may include a display that permits a user to view the integrated patient model, treatment planning steps, and/or other suitable information related to diagnosis and/or treatment planning for orthodontic treatment. In some variations, an output device may comprise a display device including at least one of a light emitting diode (LED), liquid crystal display (LCD), electroluminescent display (ELD), plasma display panel (PDP), thin film transistor (TFT), organic light emitting diodes (OLED), electronic paper/e-ink display, laser display, and/or holographic display. In some variations, an audio device may comprise at least one of a speaker, piezoelectric audio device, magnetostrictive speaker, and/or digital speaker.

The computing device may further include one or more user interfaces 280. In some variations, the user interface may comprise an input device (e.g., touch screen) and output device (e.g., display device) and be configured to receive input data. Input data may include, for example, a selection of image scan data (e.g., for rotation, cross-sectional viewing, segmenting and/or other suitable manipulation), a selection or placement of markers (e.g., to facilitate registration of surface scan data and volumetric scan data and/or facilitate model segmentation as described above) and/or other interaction with a user interface. For example, user control of an input device (e.g., keyboard, buttons, touch screen) may be received by the user interface and may then be processed by the processor and memory. Some variations of an input device may comprise at least one switch configured to generate a control signal. For example, an input device may comprise a touch surface for a user to provide input (e.g., finger contact to the touch surface) corresponding to a control signal. An input device comprising a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies. In variations of an input device comprising at least one switch, a switch may comprise, for example, at least one of a button (e.g., hard key, soft key), touch surface, keyboard, analog stick (e.g., joystick), directional pad, mouse, trackball, jog dial, step switch, rocker switch, pointer device (e.g., stylus), motion sensor, image sensor, and microphone. A motion sensor may receive user movement data from an optical sensor and classify a user gesture as a control signal. A microphone may receive audio data and recognize a user voice as a control signal.

### Exemplary Embodiments

Embodiment A1. A method for automatic registration of dental image data, the method comprising:

receiving three-dimensional intraoral surface scan data of a dentition of a patient;

receiving three-dimensional volumetric scan data of the dentition;

generating a first set of descriptors characterizing tooth crown surfaces of the dentition from the intraoral surface scan data;

generating a second set of descriptors characterizing tooth crown surfaces of the dentition from the volumetric scan data;

determining a best-fit registration transform based on the first and second sets of descriptors; and

aligning the intraoral surface scan data and the volumetric scan data based on the best-fit registration transform.

Embodiment A2. The method as in any preceding Embodiment, wherein at least one of the first set of descriptors and the second set of descriptors comprises one or more descriptors characterizing curvature of the tooth crown surfaces.

Embodiment A3. The method as in any preceding Embodiment, wherein at least one of the first and second set of descriptors comprises an estimation of a local surface normal direction, a principal curvature direction, a principal curvature value, at each of plurality of points on the tooth crown surfaces.

Embodiment A4. The method as in any preceding Embodiment, wherein determining the best-fit registration transform comprises generating one or more registration transform candidates based on matching descriptors from the first and second sets of descriptors.

Embodiment A5. The method as in any preceding Embodiment, wherein one or more of the registration transform candidates is generated based on a Hough transform.

Embodiment A6. The method as in any preceding Embodiment, wherein determining the best-fit registration transform comprises generating one or more refined registration transform candidates by applying an iterative local registration procedure to the one or more registration transform candidates.

Embodiment A7. The method as in any preceding Embodiment, wherein the iterative local registration procedure is based on an iterative closest point to plane algorithm.

Embodiment A8. The method as in any preceding Embodiment, wherein the iterative local registration procedure is based on an iterative closest point to point algorithm.

Embodiment A9. The method as in any preceding Embodiment, wherein determining the best-fit registration transform comprises generating a surface proximity measure associated with each of the one or more refined registration transform candidates, and identifying the refined registration transform candidate associated with a lowest surface proximity measure.

Embodiment A10. The method as in any preceding Embodiment, wherein determining the best-fit registration transform comprises generating a surface proximity measure associated with each of the plurality of registration transform candidates, and identifying the registration transform candidate associated with a lowest surface proximity measure.

Embodiment A11. The method as in any preceding Embodiment, wherein the intraoral surface scan data comprises optical color scan data.

Embodiment A12. The method as in any preceding Embodiment, wherein the volumetric scan data comprises cone-beam computed topography (CBCT) X-ray scan data.

Embodiment A13. The method as in any preceding Embodiment, wherein aligning the intraoral surface scan data and the volumetric scan data comprises applying the best-fit registration transform to the intraoral surface scan data to generate a transformed intraoral surface scan image.

Embodiment A14. The method as in any preceding Embodiment, wherein applying the best-fit registration transform to the intraoral surface scan data comprises applying the best-fit registration transform to a triangulated surface of the intraoral surface scan data.

Embodiment A15. The method as in any preceding Embodiment, further comprising overlaying the transformed intraoral surface scan image with a volumetric scan image associated with the volumetric scan data.

Embodiment A16. The method as in any preceding Embodiment, wherein the best-fit registration transform is configured to transform intraoral surface scan data of dentition on a first jaw of the patient.

Embodiment A17. The method as in any preceding Embodiment, further comprising determining a second best-fit registration transform, wherein the second best-fit registration transform is configured to transform intraoral surface scan data of dentition on a second jaw of the patient.

Embodiment A18. The method as in any preceding Embodiment, further comprising displaying one or more images corresponding to the aligned intraoral surface scan data and volumetric scan data.

Embodiment A19. The method as in any preceding Embodiment, further comprising generating an orthodontic treatment plan using the aligned intraoral surface scan data and volumetric scan data.

Embodiment A20. The method as in any preceding Embodiment, wherein the orthodontic treatment plan comprises defining a plurality of aligner trays with tooth-receiving cavities, wherein each aligner tray corresponds to a respective tooth arrangement.

Embodiment B1. A system for performing automatic registration of dental image data, the system comprising:

one or more processors configured to:
receive three-dimensional intraoral surface scan data of a dentition of a patient;
receive three-dimensional volumetric scan data of the dentition;
generate a first set of descriptors characterizing tooth crown surfaces of the dentition from the intraoral surface scan data;
generate a second set of descriptors characterizing tooth crown surfaces of the dentition from the volumetric scan data;
determine a best-fit registration transform based on the first and second sets of descriptors; and
align the intraoral surface scan data and the volumetric scan data based on the best-fit registration transform.

Embodiment B2. The system as in any preceding Embodiment, wherein at least one of the first set of descriptors and the second set of descriptors comprises one or more descriptors characterizing curvature of the tooth crown surfaces.

Embodiment B3. The system as in any preceding Embodiment, wherein at least one of the first and second set of descriptors comprises an estimation of a local surface normal direction, a principal curvature direction, a principal curvature value, or any combination thereof, at each of plurality of points on the tooth crown surfaces.

Embodiment B4. The system as in any preceding Embodiment, wherein the one or more processors is configured to determine the best-fit registration transform by generating one or more registration transform candidates based on matching descriptors from the first and second sets of descriptors.

Embodiment B5. The system as in any preceding Embodiment, wherein one or more of the registration transform candidates is generated based on a Hough transform.

Embodiment B6. The system as in any preceding Embodiment, wherein the one or more processors is configured to determine the best-fit registration transform by generating one or more refined registration transform candidates by applying an iterative local registration procedure to the one or more registration transform candidates.

Embodiment B7. The system as in any preceding Embodiment, wherein the iterative local registration procedure is based on an iterative closest point to plane algorithm.

Embodiment B8. The system as in any preceding Embodiment, wherein the iterative local registration procedure is based on an iterative closest point to point algorithm.

Embodiment B9. The system as in any preceding Embodiment, wherein the one or more processors is configured to determine the best-fit registration transform by generating a surface proximity measure associated with each of the one or more refined registration transform candidates, and identifying the refined registration transform candidate associated with a lowest surface proximity measure.

Embodiment B10. The system as in any preceding Embodiment, wherein the one or more processors is configured to determine the best-fit registration transform by generating a surface proximity measure associated with each of the plurality of registration transform candidates, and identifying the registration transform candidate associated with a lowest surface proximity measure.

Embodiment B11. The system as in any preceding Embodiment, wherein the intraoral surface scan data comprises optical color scan data.

Embodiment B12. The system as in any preceding Embodiment, wherein the volumetric scan data comprises cone-beam computed topography (CBCT) X-ray scan data.

Embodiment B13. The system as in any preceding Embodiment, wherein the one or more processors is configured to align the intraoral surface scan data and the volumetric scan data by applying the best-fit registration transform to the intraoral surface scan data to generate a transformed intraoral surface scan image.

Embodiment B14. The system as in any preceding Embodiment, wherein applying the best-fit registration transform to the intraoral surface scan data comprises applying the best-fit registration transform to a triangulated surface of the intraoral surface scan data.

Embodiment B15. The system as in any preceding Embodiment, wherein the one or more processors is configured to overlay the transformed intraoral surface scan image with a volumetric scan image associated with the volumetric scan data.

Embodiment B16. The method as in any preceding Embodiment, wherein the best-fit registration transform is configured to transform intraoral surface scan data of dentition on a first jaw of the patient.

Embodiment B17. The method as in any preceding Embodiment, wherein the one or more processors is configured to determine a second best-fit registration transform, wherein the second best-fit registration transform is configured to transform intraoral surface scan data of dentition on a second jaw of the patient.

Embodiment B18. The system as in any preceding Embodiment, further comprising a display configured to display one or more images corresponding to the aligned intraoral surface scan data and volumetric scan data.

Embodiment B19. The system as in any preceding Embodiment, wherein the one or more processors is configured to generate an orthodontic treatment plan using the aligned intraoral surface scan data and volumetric scan data.

Embodiment B20. The system as in any preceding Embodiment, wherein the orthodontic treatment plan comprises defining a plurality of aligner trays with tooth-receiving cavities, wherein each aligner tray corresponds to a respective tooth arrangement.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A method for automatic registration of dental image data, the method comprising:
receiving three-dimensional intraoral surface scan data of a dentition of a patient;
receiving three-dimensional volumetric scan data of the dentition;
generating a first set of descriptors characterizing tooth crown surfaces of the dentition from the intraoral surface scan data;
generating a second set of descriptors characterizing tooth crown surfaces of the dentition from the volumetric scan data;
determining a best-fit registration transform based on the first and second sets of descriptors; and
aligning the intraoral surface scan data and the volumetric scan data based on the best-fit registration transform.

2. The method of claim 1, wherein at least one of the first set of descriptors and the second set of descriptors comprises one or more descriptors characterizing curvature of the tooth crown surfaces.

3. The method of claim 2, wherein at least one of the first and second set of descriptors comprises an estimation of a local surface normal direction, a principal curvature direction, a principal curvature value, at each of plurality of points on the tooth crown surfaces.

4. The method of claim 1, wherein determining the best-fit registration transform comprises generating one or more registration transform candidates based on matching descriptors from the first and second sets of descriptors.

5. The method of claim 4, wherein one or more of the registration transform candidates is generated based on a Hough transform.

6. The method of claim 4, wherein determining the best-fit registration transform comprises generating one or more refined registration transform candidates by applying an iterative local registration procedure to the one or more registration transform candidates.

7. The method of claim 6, wherein the iterative local registration procedure is based on an iterative closest point to plane algorithm.

8. The method of claim 6, wherein the iterative local registration procedure is based on an iterative closest point to point algorithm.

9. The method of claim 6, wherein determining the best-fit registration transform comprises generating a surface proximity measure associated with each of the one or more refined registration transform candidates, and identifying the refined registration transform candidate associated with a lowest surface proximity measure.

10. The method of claim 4, wherein determining the best-fit registration transform comprises generating a surface proximity measure associated with each of the plurality of registration transform candidates, and identifying the registration transform candidate associated with a lowest surface proximity measure.

11. The method of claim 1, wherein the intraoral surface scan data comprises optical color scan data.

12. The method of claim 1, wherein the volumetric scan data comprises cone-beam computed topography (CBCT) X-ray scan data.

13. The method of claim 1, wherein aligning the intraoral surface scan data and the volumetric scan data comprises applying the best-fit registration transform to the intraoral surface scan data to generate a transformed intraoral surface scan image.

14. The method of claim 13, wherein applying the best-fit registration transform to the intraoral surface scan data comprises applying the best-fit registration transform to a triangulated surface of the intraoral surface scan data.

15. The method of claim 13, further comprising overlaying the transformed intraoral surface scan image with a volumetric scan image associated with the volumetric scan data.

16. The method of claim 1, wherein the best-fit registration transform is configured to transform intraoral surface scan data of dentition on a first jaw of the patient.

17. The method of claim 16, further comprising determining a second best-fit registration transform, wherein the second best-fit registration transform is configured to transform intraoral surface scan data of dentition on a second jaw of the patient.

18. The method of claim 1, further comprising displaying one or more images corresponding to the aligned intraoral surface scan data and volumetric scan data.

19. The method of claim 1, further comprising generating an orthodontic treatment plan using the aligned intraoral surface scan data and volumetric scan data.

20. The method of claim 19, wherein the orthodontic treatment plan comprises defining a plurality of aligner trays with tooth-receiving cavities, wherein each aligner tray corresponds to a respective tooth arrangement.

21. A system for performing automatic registration of dental image data, the system comprising:
one or more processors configured to:
receive three-dimensional intraoral surface scan data of a dentition of a patient;
receive three-dimensional volumetric scan data of the dentition;
generate a first set of descriptors characterizing tooth crown surfaces of the dentition from the intraoral surface scan data;
generate a second set of descriptors characterizing tooth crown surfaces of the dentition from the volumetric scan data;
determine a best-fit registration transform based on the first and second sets of descriptors; and
align the intraoral surface scan data and the volumetric scan data based on the best-fit registration transform.

22. The system of claim 21, wherein at least one of the first set of descriptors and the second set of descriptors comprises one or more descriptors characterizing curvature of the tooth crown surfaces.

23. The system of claim 22, wherein at least one of the first and second set of descriptors comprises an estimation of a local surface normal direction, a principal curvature direction, a principal curvature value, or any combination thereof, at each of plurality of points on the tooth crown surfaces.

24. The system of claim 21, wherein the one or more processors is configured to determine the best-fit registration transform by generating one or more registration transform candidates based on matching descriptors from the first and second sets of descriptors.

25. The system of claim 24, wherein one or more of the registration transform candidates is generated based on a Hough transform.

26. The system of claim 24, wherein the one or more processors is configured to determine the best-fit registration transform by generating one or more refined registration transform candidates by applying an iterative local registration procedure to the one or more registration transform candidates.

27. The system of claim 26, wherein the iterative local registration procedure is based on an iterative closest point to plane algorithm.

28. The system of claim 26, wherein the iterative local registration procedure is based on an iterative closest point to point algorithm.

29. The system of claim 26, wherein the one or more processors is configured to determine the best-fit registration transform by generating a surface proximity measure associated with each of the one or more refined registration transform candidates, and identifying the refined registration transform candidate associated with a lowest surface proximity measure.

30. The system of claim 24, wherein the one or more processors is configured to determine the best-fit registration transform by generating a surface proximity measure associated with each of the plurality of registration transform candidates, and identifying the registration transform candidate associated with a lowest surface proximity measure.

31. The system of claim 21, wherein the intraoral surface scan data comprises optical color scan data.

32. The system of claim 21, wherein the volumetric scan data comprises cone-beam computed topography (CBCT) X-ray scan data.

33. The system of claim 21, wherein the one or more processors is configured to align the intraoral surface scan data and the volumetric scan data by applying the best-fit registration transform to the intraoral surface scan data to generate a transformed intraoral surface scan image.

34. The system of claim 33, wherein applying the best-fit registration transform to the intraoral surface scan data comprises applying the best-fit registration transform to a triangulated surface of the intraoral surface scan data.

35. The system of claim 33, wherein the one or more processors is configured to overlay the transformed intraoral surface scan image with a volumetric scan image associated with the volumetric scan data.

36. The method of claim 1, wherein the best-fit registration transform is configured to transform intraoral surface scan data of dentition on a first jaw of the patient.

37. The method of claim 36, wherein the one or more processors is configured to determine a second best-fit registration transform, wherein the second best-fit registration transform is configured to transform intraoral surface scan data of dentition on a second jaw of the patient.

38. The system of claim 21, further comprising a display configured to display one or more images corresponding to the aligned intraoral surface scan data and volumetric scan data.

39. The system of claim 21, wherein the one or more processors is configured to generate an orthodontic treatment plan using the aligned intraoral surface scan data and volumetric scan data.

40. The system of claim 39, wherein the orthodontic treatment plan comprises defining a plurality of aligner trays with tooth-receiving cavities, wherein each aligner tray corresponds to a respective tooth arrangement.
